(19) United States
(12) Patent Application Publication
(10) Pub. No.: US 2013/0334042 A1
(43) Pub. Date: Dec. 19, 2013

(54) PATHOGEN SENSOR

(71) Applicants: Bruce Donaldson Grieve, Greater Manchester (GB); Sarah Perfect, Bracknell (GB); Sophie Weiss, Greater Manchester (GB)

(72) Inventors: Bruce Donaldson Grieve, Greater Manchester (GB); Sarah Perfect, Bracknell (GB); Sophie Weiss, Greater Manchester (GB)

(73) Assignee: SYNGENTA LIMITED, Guildford Surrey (GB)

(21) Appl. No.: 13/991,088

(22) PCT Filed: Dec. 5, 2011
(86) PCT No.: PCT/EP2011/071686

§ 371 Date: Aug. 20, 2013

(30) Foreign Application Priority Data
Dec. 6, 2010 (GB) 1020619.1

(51) Int. Cl. C12Q 1/04; C12Q 1/00

(52) U.S. Cl. CPC C12Q 1/04; C12Q 1/005
USPC 204/403.14; 204/403.01

(57) ABSTRACT

A pathogen sensor comprising a growth medium upon which and/or within which a pathogen may grow, the growth medium comprising nutrients which facilitate growth of the pathogen, wherein the pathogen sensor further comprises an electronic detection apparatus configured to detect an electrochemical change mediated by the pathogen.

The present invention relates to a pathogen sensor.

Pathogens are agents that cause infection or disease, especially microorganisms such as bacteria, protozoan, viruses and fungi.

Phytopathology or plant pathology relates to the diagnosis and management of plant diseases caused by infection agents or diseases that attack plants and environmental conditions. Organisms that cause diseases in plants include for example: fungi (including molds and yeasts), viruses, oomycetes, bacteria, viroids, phytoplasmas, protozoa, nematodes and parasitic plants.

In farming it is conventional to monitor the health of a crop through visual inspection of the crop. Growth of a pathogen on a crop may be identified via this visual inspection, whereupon a suitable agent such as a fungicide may be applied to the crop. In addition to visual inspection of the crop, a farmer may take into account environmental conditions such as the weather (including predicted future environmental conditions). Although this approach may work in some instances it is desirable to provide an apparatus which is capable of indicating that a pathogen is growing or is likely to be growing in a crop.

According to a first aspect of the invention there is provided a pathogen sensor comprising a growth medium upon which and/or within which a pathogen may grow, the growth medium being provided with nutrients which facilitate growth of the pathogen, wherein the pathogen sensor further comprises an electronic detection apparatus configured to detect an event mediated by the pathogen.

The event mediated by the pathogen may be the production of a chemical or biological agent. The chemical or biological agent may be one of the following: an organic acid, a nucleic acid, a protein, an enzyme, a toxin, a hormone, a metabolite, a peptide, a carbohydrate or a lipid.

The chemical agent to be detected may be oxalic acid. Oxalic acid is an organic compound with the formula H₂C₂O₄. This colourless solid is a dicarboxylic acid and is about 3,000 times stronger than acetic acid. Oxalic acid is a reducing agent and its conjugate base, known as oxalate (C₂O₄²⁻), is a chelating agent for metal cations. Typically oxalic acid occurs as the dihydrate with the formula C₂O₄H₂.2H₂O.

Oxalic acid and derivatives thereof such as oxalates are present in many plants. Consequently, oxalic acid, and salts or derivatives thereof is a suitable candidate for detection in a pathogen sensor of the present invention.

The electronic detection apparatus may be configured to detect an electrochemical change in the growth medium.

The electronic detection apparatus may comprise an enzyme that interacts with the chemical or biological agent, the interaction leading to an electronically detectable signal. The interaction may generate an electroactive species or lead to the generation of an electroactive species. The electronic detection apparatus may further comprise an electrode configured to detect the presence of the electroactive species.

The electrode may have been modified by a biochemical and/or chemical recognition element. This may for example include incorporating an enzyme, antibody, DNA or chemical species into the electrode which may enhance or change the electrochemical response of the electrode.

The enzyme may be located in a biocompatible polymer. The biocompatible polymer may be a hydrophilic polymer, or may be formed from hydrophilic monomers. The enzyme may be immobilised on a surface of the electrode. The enzyme may be immobilised in a biocompatible polymer. The enzyme may be oxalate oxidase. The pathogen sensor may further comprise horseradish peroxidase.

Horseradish peroxidase is a 44,173.9-dalton glycoprotein with four lysine residues for conjugation to for example a labeled molecule. It produces a coloured, fluorimetric, or luminescent derivative of the labeled molecule when incubated with a proper substrate, allowing it to be detected and quantified.

The pathogen sensor may further comprise a nutrient reservoir which is configured to provide a supply of nutrients to the growth medium. The nutrient reservoir may be configured to supply nutrients to the growth medium for a period which is longer than 10 hours.

The growth medium may be a nutrient liquid.

The pathogen sensor may further comprise a fluid reservoir which is configured to provide a supply of fluid to the growth medium to prevent dehydration of the growth medium. The fluid reservoir may be configured to supply fluid to the growth medium for a period which is longer than 10 hours. The nutrient reservoir and the fluid reservoir may be the same reservoir.

The growth medium may have one or more properties which mimic an entity upon which and/or within which the pathogen will grow. The one or more properties may include at least one of the following: lighting of the growth medium, humidity or moisture conditions at the growth medium, pH conditions at the growth medium, the orientation of the growth medium, and the temperature of the growth medium.

The entity may be a plant.

The growth medium may be provided with one or more fungicides, antibiotics or antimicrobials which do not prevent growth of the pathogen.

The pathogen may be a fungal pathogen. The pathogen may be Sclerotinia sclerotiorum. Sclerotinia sclerotiorum is a plant pathogenic fungus that can cause a disease called white mold if conditions are correct. S. sclerotiorum can also be known as cottony rot, watery soft rot, stem rot, drop, crown rot and blossom blight. A key characteristic of this pathogen is its ability to produce black resting structures known as sclerotia and white fuzzy growths of mycelium on the plant it infects. These sclerotia give rise to a fruiting body in the spring that produces spores in a sac, which is why fungi in this class are called sac fungi (Ascomycetes). This pathogen can occur on many continents and has a wide host range of plants. When S. sclerotiorum is onset in the field by favorable environmental conditions, losses can be great.

Sclerotinia sclerotiorum proliferates in moist environments. Under moist field conditions, S. sclerotiorum is capable of completely invading a plant host, colonizing nearly all of the plant's tissues with mycelium. Optimal temperatures for growth range from 15 to 21 degrees Celsius. Under wet conditions, S. sclerotiorum will produce an abundance of mycelium and sclerotia.

The pathogen may be a bacterial pathogen. The pathogen may be from the Burkholderia genus.

According to a second aspect of the invention there is provided a sensor apparatus which comprises the pathogen sensor according to the first aspect of the invention and which further comprises measurement electronics configured to receive a signal from the electronic detection apparatus and to generate an output if the signal indicates that an event mediated by the pathogen has occurred. The sensor apparatus may include any of the above features of the pathogen sensor.

The pathogen sensor may be releasably engageable with the sensor apparatus such that the pathogen sensor may be replaced with another pathogen sensor. The pathogen sensor may be one of a plurality of pathogen sensors provided in a cartridge which is releasably engageable with the sensor apparatus.

According to a third aspect of the invention there is provided a method of detecting a pathogen comprising providing nutrients which facilitate growth of the pathogen on and/or in a growth medium for a period which is sufficiently long to allow an event mediated by the pathogen to occur, then using an electronic detection apparatus to detect the mediated event. The growth environment may be a favourable growth environment. The favourable growth environment may be an environment which facilitates growth of the pathogen at a rate which is faster than the rate at which the pathogen will grow on a plant or other entity adjacent to which the pathogen sensor is provided.

The event mediated by the pathogen may be the production of a chemical or biological agent. The chemical or biological agent may be one of the following: an organic acid, a nucleic acid, a protein, an enzyme, a toxin, a hormone, a metabolite, a peptide, a carbohydrate or a lipid. The chemical agent may be oxalic acid.

The electronic detection apparatus may detect an electrochemical change in the growth medium.

The electronic detection apparatus may comprise an enzyme which interacts with the chemical or biological agent, the interaction leading to an electronically detectable signal. The interaction may lead to the generation of an electroactive species. The method may further comprise detecting the presence of the electroactive species using an electrode.

The enzyme may be oxalate oxidase which catalyses the production of hydrogen peroxide from the oxalic acid. The pathogen sensor may further comprise horseradish peroxidase which reduces the hydrogen peroxide.

Detecting the presence of the electroactive species using the electrode may comprise applying a first potential and a second different potential to the electrode and measuring the resulting current.

The method may further comprise supplying nutrients to the growth medium for a period which is longer than 10 hours. The method may further comprise supplying fluid to the growth medium for a period which is longer than 10 hours.

The growth medium may have one or more properties which mimic an entity upon which and/or within which the pathogen will grow. The one or more properties may include at least one of the following: lighting of the growth medium, humidity or moisture conditions at the growth medium, pH conditions at the growth medium, the orientation of the growth medium, and the temperature of the growth medium.

The pathogen may be a fungal pathogen. The pathogen may be Sclerotinia Sclerotiorum. The pathogen may be a bacterial pathogen. The pathogen may be from the Burkholderia genus.

The method may comprise exposing the growth medium to the air and monitoring for the mediated event and then subsequently exposing a second growth medium to the air and monitoring for the mediated event.

A method of detecting the presence of a pathogen in the environment comprising exposing to air the pathogen sensor of any preceding paragraph and monitoring for the mediated event.

The pathogen sensor may be provided in a crop or adjacent to a crop, such that the method provides an indication of whether a pathogen is growing in the crop or is likely to be growing in the crop. The pathogen sensor may be provided in a storage area in which a crop is stored after the crop has been harvested (e.g. a warehouse or barn).

The pathogen sensor may be one of a plurality of pathogen sensors distributed over an area. The method may comprise analysing outputs from the pathogen sensors to obtain information regarding the progress of the pathogen through the area.

Analysis of information provided from the pathogen sensor may be combined with analysis of information provided from one or more sensors which sense one or more of: temperature, humidity, wind direction, wind speed, pressure sensor and ambient light.

According to a fourth aspect of the invention there is provided a pathogen sensor comprising a growth medium upon which and/or within which a pathogen may grow, the growth medium comprising nutrients which facilitate growth of the pathogen, wherein the pathogen sensor further comprises an electronic detection apparatus configured to detect an electrochemical change mediated by the pathogen.

The electrochemical change may be caused by a chemical or biological agent produced by the pathogen.

The growth medium may be a liquid media which contains potato dextrose broth. The growth medium may be potato dextrose agar.

The pathogen may be from the Sclerotinia species. The pathogen may be Sclerotinia Sclerotiorum.

According to a fifth aspect of the invention there is provided a sensor apparatus which comprises the pathogen sensor of any preceding aspect of the invention, and further comprises measurement electronics configured to receive a signal from the electronic detection apparatus and to generate an output if the signal is indicative of an electrochemical change mediated by the pathogen.

The sensor apparatus may further comprise a control apparatus which is configured to expose the pathogen sensor to the air, incubate the pathogen sensor for a predetermined period of time, and then use the electronic detection apparatus to monitor for the electrochemical change.

The sensor apparatus may further comprise a puncturing apparatus configured to puncture a barrier which separates the growth medium from the electrode.

According to a sixth aspect of the invention method of detecting a pathogen comprising providing nutrients which facilitate growth of the pathogen on and/or in a growth medium for a period which is sufficiently long to allow a pathogen to mediate an electrochemical change, then using an electronic detection apparatus to detect the electrochemical change.

The electrochemical change may be caused by a chemical or biological agent produced by the pathogen.

According to a seventh aspect of the invention there is provided a sensor apparatus which comprises the pathogen sensor of any preceding claim and further comprises measurement electronics configured to receive a signal from the electronic detection apparatus and to generate an output if the signal is indicative of an electrochemical change mediated by the pathogen.

The sensor apparatus may further comprise a control apparatus which is configured to expose the pathogen sensor to the air, incubate the pathogen sensor for a predetermined period of time, and then use the electronic detection apparatus to monitor for the electrochemical change.

The sensor apparatus may further comprise a puncturing apparatus configured to puncture a barrier which separates the growth medium from the electrode.

According to an eighth aspect of the invention there is provided use of a pathogen sensor according to any preceding aspect or a sensor apparatus according to any preceding aspect for detecting an electrochemical change in crops arising from the presence of one or more of: fungi (including molds and yeasts), viruses, oomycetes, bacteria, viroids, phytoplasmas, protozoa, nematodes and parasitic plants on the crop.

According to a ninth aspect of the invention there is provided use of a pathogen sensor as described in relation to any of preceding aspect or a sensor apparatus according to any preceding aspect in the treatment of wheat and barley.

Features of different aspects of the invention may be combined with one another.

Specific embodiments of the invention will now be described by way of example only, with reference to the accompanying figures in which:

FIG. 1 shows schematically in cross-section a pathogen sensor according to an embodiment of the invention;

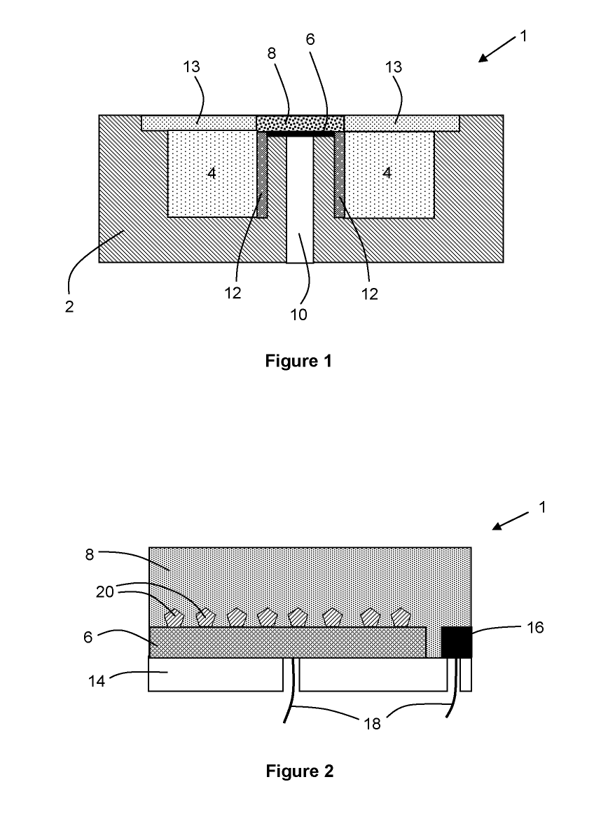

FIG. 2 shows schematically in cross-section a pathogen sensor according to an alternative embodiment of the invention;

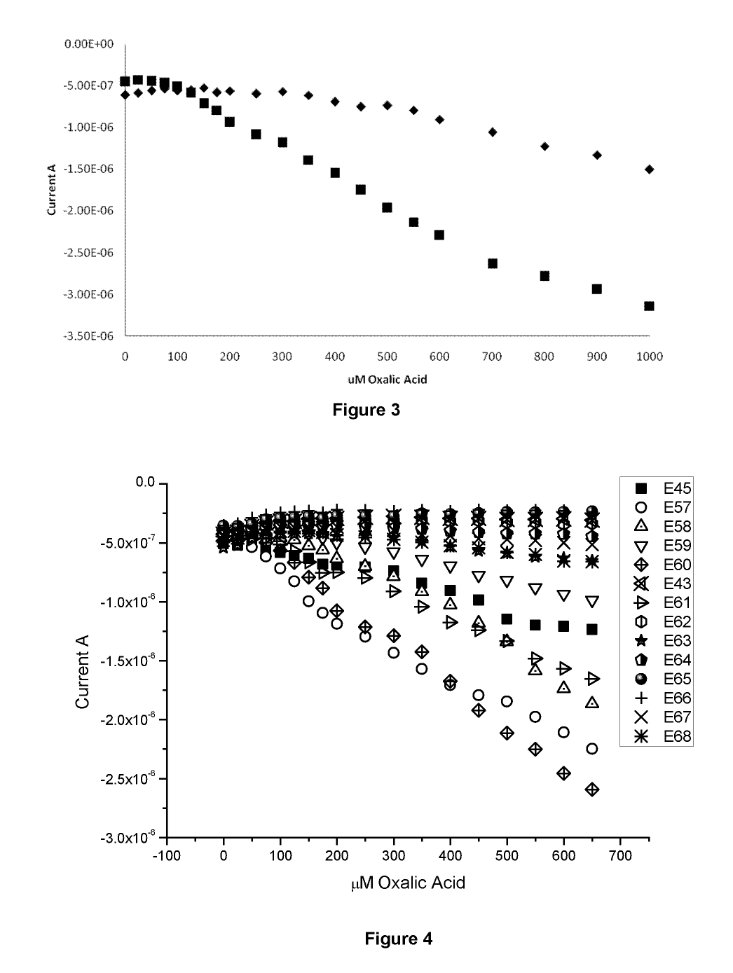

FIG. 3 is a graph which demonstrates that oxalic acid may be detected using a pathogen sensor according to an embodiment of the invention;

FIG. 4 is a graph which demonstrates that oxalic acid may be detected using a pathogen sensor according to an embodiment of the invention, including particular growth media;

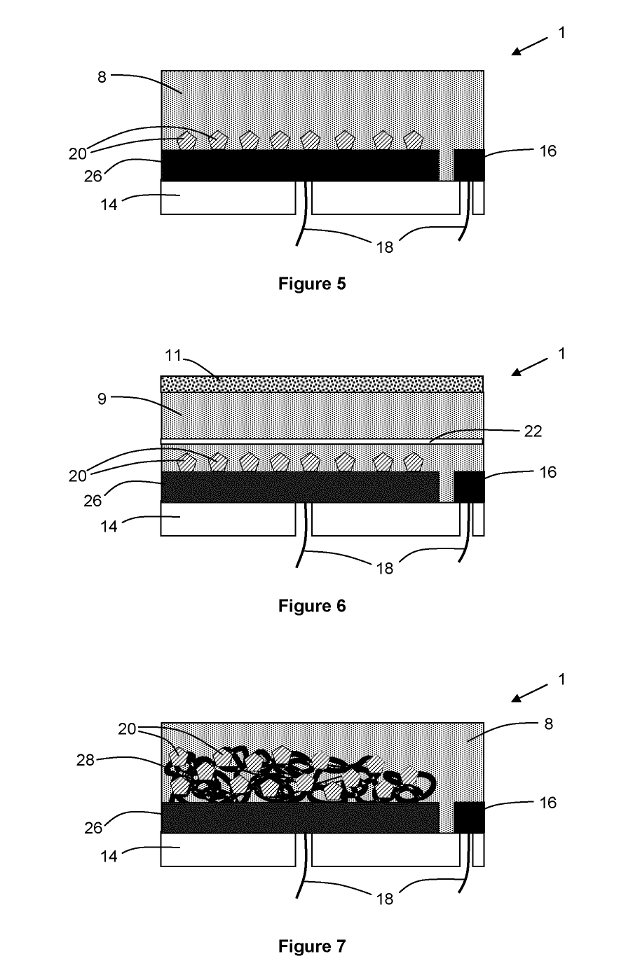

FIG. 5 shows schematically in cross-section a pathogen sensor according to a further alternative embodiment of the invention;

FIG. 6 shows schematically in cross-section a pathogen sensor according to a further alternative embodiment of the invention;

FIG. 7 shows schematically in cross-section a pathogen sensor according to a further alternative embodiment of the invention;

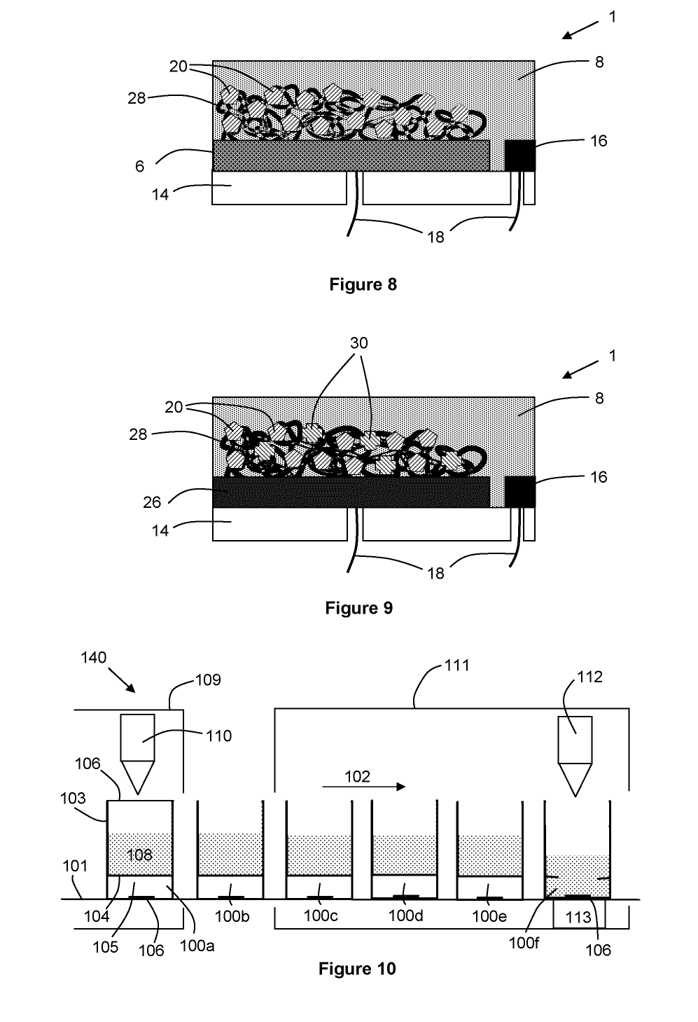

FIG. 8 shows schematically in cross-section a pathogen sensor according to a further alternative embodiment of the invention;

FIG. 9 shows schematically in cross-section a pathogen sensor according to a further alternative embodiment of the invention;

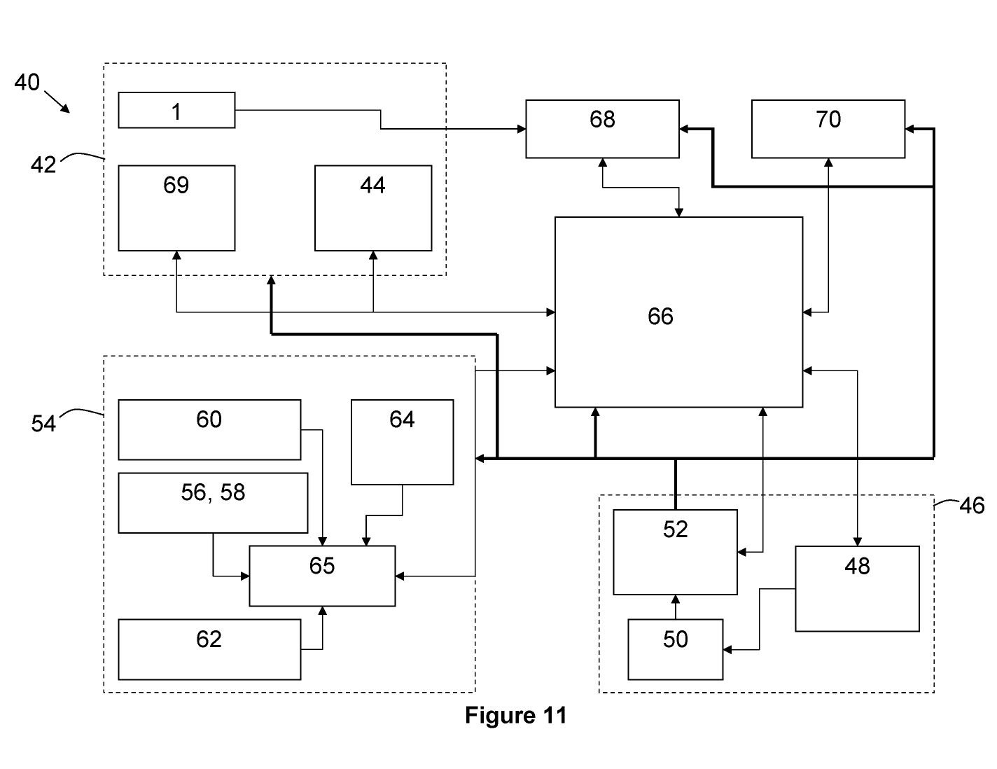

FIG. 10 shows schematically a sensor apparatus according to an embodiment of the invention; and

FIG. 11 shows schematically an alternative sensor apparatus according to an embodiment of the invention.

FIG. 1 shows schematically in cross-section a pathogen sensor 1 according to an embodiment of the invention. The pathogen sensor 1 comprises a support structure 2, a nutrient reservoir 4, an electrode 6 and a gel 8. The nutrient reservoir 4 is annular, and extends around a central portion of the support structure 2. The support structure may for example be formed from plastic or some other suitable material. The gel 8 is provided on top of the electrode 6 and has an upper surface which is exposed to the atmosphere. The electrode 6 is supported on a substrate (not shown). A cylindrical channel 10 extends downwardly from the electrode 6 and may accommodate a wire or wires (not shown) which are connected to the electrode. Additional electrodes such as a reference electrode and a counter electrode (not shown) may be provided. A one-way membrane 12 is provided around an outer wall of the cylindrical channel 10, thereby forming an inner wall of the nutrient reservoir 4. The one-way membrane 12 is configured such that water based nutrients may pass through it from the nutrient reservoir 4 and may then travel to the gel 8. The one-way membrane 12 does not allow the water based nutrients to flow from the gel 8 into the liquid nutrient reservoir 4. An upper surface of liquid nutrient reservoir 4 is covered by an annular gas permeable sealing layer 13. The gas permeable sealing layer 13 allows gas (e.g. air) to pass into the nutrient reservoir 4 and thereby prevents a pressure drop occurring when water based nutrients leave the nutrient reservoir. In addition, the gas permeable sealing layer 13 allows oxygen to be absorbed into the water based nutrients. This is desirable because oxygen is one of the components of an electrochemical reaction which will take place in the pathogen sensor when a pathogen is present (as is described further below).

The gel 8 may be a non-water based gel which is configured to adhere to the surface of the electrode 6. The gel 8 may be considered to be an example of a growth medium upon which and/or within which a pathogen may grow. The gel 8 may for example be potato dextrose agar (PDA). The gel 8 absorbs water based nutrients through the one-way membrane 12 via osmotic pressure. The osmotic pressure is generated by evaporation of liquid from the gel 8. The membrane 12 may deliver the water based nutrients to the gel 8 via a wicking action. The membrane 12 may for example be a polyethylene material which is sulphonated on one side to make it hydrophilic and which is naturally hydrophobic on the other side (similar to a membrane used in a diaper). Alternatively, functional groups other than sulphonates may be applied to one side of the polyethylene material to ensure one side of the material is hydrophilic. The functional groups may be for example, but are not limited to, hydroxyl, carboxyl, amino, phosphate and sulfhydryl groups. The water based nutrients may for example comprise potato dextrose broth (PDB), a sunflower derived nutrient or some other nutrient.

The one-way membrane 12 provides a supply of water based nutrients to the gel 8 until the nutrient reservoir 4 is empty. Providing a supply of nutrients to the gel 8 is advantageous because it replaces nutrients as they are used by a pathogen growing on the pathogen sensor. A further advantage of providing the supply of water based-nutrients is that this ensures that the gel 8 remains hydrated. If the gel 8 were to dry out then growth of a pathogen on the gel could be inhibited. In addition, the ability of the pathogen sensor 1 to detect the presence of a pathogen could be compromised if the gel 8 were to dry out.

The pathogen sensor 1 may be provided with a seal (not shown) on its upper surface which acts to prevent the gel 8 (and optionally the nutrient reservoir 4) being exposed to air until operation of the pathogen sensor is desired, the seal being removed in order to initiate operation of the pathogen sensor. This prevents evaporation of water from the gel 8 occurring before operation of the pathogen sensor is desired and hence the drying out of the gel.

The gel 8 may for example be 500-1000 microns thick and may for example have a diameter of 3 mm. The electrode 6 may for example have a thickness of 100 microns and may for example have a diameter of 2 mm. The nutrient reservoir 4 may for example be 1-2 mm deep and may for example have a diameter of 10 mm. These dimensions are given merely as examples, and the gel, electrode and nutrient reservoir may have other dimensions.

An oxalate oxidase enzyme may be provided on the electrode 6 or in the vicinity of the electrode.

In enzymology, an oxalate oxidase is an enzyme that catalyzes the chemical reaction of oxalate to carbon dioxide and hydrogen peroxide as illustrated below.

oxalate+O₂+2H⁺ [symbol] 2CO₂+H₂O₂

The substrates of this enzyme are therefore oxalate (derived from oxalic acid), oxygen (O₂), and hydrogen ions (H⁺), whereas the two products are CO₂ and H₂O₂.

Oxalate oxidases belong to the family of oxidoreductases, specifically those enzymes acting on an aldehyde or oxo group of a donor with oxygen as an acceptor. The systematic name of this enzyme class is oxalate:oxygen oxidoreductase. However, other common names include for example aero-oxalo dehydrogenase, and oxalic acid oxidase. This enzyme participates in glyoxylate and dicarboxylate metabolism.

The oxalate oxidase is provided in such a manner that it retains its activity and stability. As explained below, oxalate oxidase enzymes will catalyse the generation of hydrogen peroxide when oxalic acid/oxalate and oxygen are present at the oxalate oxidase. The presence of the hydrogen peroxide may be detected via the electrode 6. The detected hydrogen peroxide may indicate that a pathogen has grown on the gel 8 and has released oxalic acid (some plant pathogens release oxalic acid when they grow). Thus, the oxalate oxidase may be considered to form part of an electronic detection apparatus which detects the oxalic acid. The electrode may also be considered to form part of the electronic detection apparatus.

The pathogen sensor 1 may be provided at a location where it is desired to monitor for the presence of a pathogen. The seal may be removed from the pathogen sensor, thereby exposing the gel 8 to the atmosphere. Removing the seal also exposes the water based nutrients in the nutrient reservoir 4 to the atmosphere via the gas permeable sealing layer 13. Water based nutrients are drawn by the gel 8 through the one-way membrane 12, thereby ensuring that the gel remains supplied with water based nutrients and remains hydrated. This facilitates growth of a pathogen which may arrive at the sensor and then germinate and grow. The pathogen may grow for a period of time on or in the gel using the water based nutrients provided from the nutrient reservoir 4. The pathogen may then release oxalic acid, the catalytic breakdown of the oxalic acid being detected by the electrode 6 as is explained further below. The release of oxalic acid and the subsequent catalytic breakdown of the oxalic acid may be considered to be an event which is mediated by the pathogen.

It may take a considerable period of time (e.g. 10 hours to 2 days, 4 days or more) for the pathogen to grow sufficiently that it may mediate the event (e.g. the release and catalytic breakdown of oxalic acid). It is desirable that the pathogen sensor 1 is capable of operating for a period of time which is longer than the period required for the pathogen to grow and mediate the event. The pathogen sensor may for example be capable of operating for 10 hours, 24 hours, 2 days, 3 days, 4 days or more. The pathogen sensor may thus for example be capable of providing a supply of nutrients to the gel 8 for 10 hours, 24 hours, 2 days, 3 days, 4 days or more, and may be capable of keeping the gel 8 hydrated for 10 hours, 24 hours, 2 days, 3 days, 4 days or more.

When the mediated event takes place it is detected by the electrode 6 as is explained further below. This indicates that the pathogen is present and is growing. When the presence of the pathogen has been detected, measurement electronics connected to the pathogen sensor may provide an output indicating the presence of the pathogen. This for example allows a farmer to take appropriate measures to protect from the pathogen crops which are located in the vicinity of the pathogen sensor.

The pathogen sensor 1 may for example be configured to detect Sclerotinia Sclerotiorum. Where this is the case the pathogen sensor provides a growth medium (the gel 8) upon and/or within which S. sclerotiorum may grow, and provides nutrients which nourish the S. sclerotiorum over a period of time which is sufficient to allow the S. sclerotiorum to grow to an extent that it will produce oxalic acid. In addition, the nutrients may facilitate the production of oxalic acid by the S. sclerotiorum. The nutrients may facilitate growth of S. sclerotiorum via metabolic pathways which provide more oxalic acid production than alternative metabolic pathways (the alternative metabolic pathways producing less oxalic acid). Selective fungicides, antibiotics or antimicrobials may be incorporated in the pathogen sensor to inhibit the growth of other microorganisms which may inhibit S. sclerotiorum growth and/or produce oxalic acid or some other interferent electroactive species.

The pathogen sensor may detect S. sclerotiorum by detecting oxalic acid released by the S. sclerotiorum. Detection of oxalic acid may be used in the pathogen sensor to detect the presence of other fungal pathogens which produce oxalic acid. Examples of such fungal pathogens include: Ascomycetes, and may include Aspergillus fonsecaeus, Aspergillus niger, Botrytis cinerea, Cryphonectria parasitica, Saccharomyces cerevisiae, Saccharomyces hansenii, Penicillium bilaii, Penicillium oxalicum, Sclerotium cepivorum, Sclerotium delphinii, Sclerotium glucanicum, Sclerotium rolfsii, Sclerotinia sclerotiorum, Sclerotinia trifoliorum. Examples also include Deuteromycetes, and may include Cristulariella pyramidalis, Leucostoma cincta and Leucostoma persoonii. Examples also include Basidiomycetes, and may include Rhizoctonia solani, Postia placenta, Fomitopsis palustris and Woffiporia cocos. Examples also include other wood rotting fungal species that secrete oxalic acid.

Measurement electronics (not shown) are configured to apply a potential at the electrode 6 which is stepped between a first value at which no electroactive reactions occur and a second value at which an electroactive reaction occurs when hydrogen peroxide is present at the electrode. The change of potential from the first value to the second value and back again may for example be applied intermittently. The detection methodology used by the electronic detection apparatus may be referred to as chronoamperometry, and may be considered to be an example of electrochemical detection. The hydrogen peroxide is generated as a result of the breakdown of oxalic acid released by the pathogen (e.g. S. sclerotiorum), the generation of the hydrogen peroxide taking place in the presence of oxygen and the oxalate oxidase provided at the electrode 6. The potential change at the electrode 6 caused by the hydrogen peroxide results in a characteristic charging and decay current which is proportional (e.g. directly proportional) to the concentration of the hydrogen peroxide at the electrode.

The second value of the potential applied to the electrode 6 (i.e. the value at which the electroactive reaction occurs) may be chosen for optimal electron transfer to the hydrogen peroxide, thereby maximising the current caused by the hydrogen peroxide. Similarly, the time period during which the second potential value is applied to the electrode may be chosen to facilitate detection of the hydrogen peroxide. An explanation of this detection methodology may be found in Electroanalysis by C. M. A. Brett and A. M. Oliveira Brett, 1998, which is herein incorporated by reference.

An alternative embodiment of the invention is shown schematically in cross-section in FIG. 2. In the embodiment shown in FIG. 2, a working electrode 6 and a reference electrode 16 are provided, the reference electrode being separated from the working electrode. The working electrode 6 may for example have a surface area of 3 mm² and the reference electrode 16 may for example have a surface area of 0.5 mm². The working electrode 6 and reference electrode 16 are provided on a substrate 14. The substrate 14 may for example be 50 mm long and 10 mm wide. Wires 18 extend from the working electrode 6 and the reference electrode 16, the wires passing through openings in the substrate 14 to measurement electronics (not shown). A nutrient liquid 8 is provided over the electrodes 6, 16. The nutrient liquid 8 is held in place by walls (not shown), with an upper surface of the nutrient liquid being exposed to the atmosphere. The nutrient liquid 8 is an example of a growth medium.

An oxalate oxidase 20 is attached to the working electrode 6. The oxalate oxidase was generated in a purified form by taking the oxalate oxidase gene from barley (Hordeum vulgare) and expressing it in a Pichia (a type of yeast) expression system. In more detail, the method used to obtain the purified oxalate oxidase is as follows: the mature Hordeum vulgare (Barley) oxalate oxidase open reading frame (GenBank reference no. 289356) was codon-optimised for expression in Pichia pastoris and synthesised as an XhoI/NotI fragment designed to create an in-frame fusion with the yeast α-mating factor when cloned into the vector pPICZαA (Invitrogen). The assembled oxalate oxidase extracellular expression vector was used to transform competent P. pastoris according to published protocols by Whittaker M M and Whittaker J W, Journal of Biological Inorganic Chemistry, 2002 January; 7(1-2):136-45 (herein incorporated by reference). A large scale (5 litres) high density X33 (a strain of Pichia pastoris) fermentation was carried out as described in the same paper. 120 mg of protein was purified from the supernantant broth using cation exchange chromatography and size exclusion chromatography, which exhibited enzymatic activity in a colorimetric assay. Oxalate oxidase protein identification was confirmed by peptide mass fingerprinting (MALDI-TOF) and whole mass spectroscopy using Q-ToF.

The oxalate oxidase was stored as a lyophilised powder, and was prepared as a 1 mg/ml aqueous solution in a 2× buffer and a 2× stabiliser solution. The buffer was 100 mM succinic acid, 200 mM KCl, pH 3.8. Q209011D10, which is available from Applied Enzyme Technology of Pontypool, United Kingdom, may be used as the stabiliser solution. Other suitable buffers and stabilisers (e.g. sugars and polyelectrolytes) may be used.

The oxalate oxidase solution was pipetted onto the working electrode 6 (e.g. 10 μl of oxalate oxidase solution; other quantities of solution may be used). The solution was then allowed to dry completely (e.g. drying for several hours). This dried version of the oxalate oxidase is stable at room temperature for many weeks. The nutrient liquid 8 was subsequently provided on top of the working electrode 6. When this was done the oxalate oxidase rehydrated and became active again but stayed on the surface of the working electrode 6 (the oxalate oxidase was adsorbed to the working electrode). Rehydration of the oxalate oxidase was necessary in order to allow the oxalate oxidase to catalyse the generation of hydrogen peroxide when oxalic acid/oxalate and oxygen are present.

An alternative oxalate oxidase which comprises a partially purified form of oxalate oxidase derived from barley seedlings may be used. However, this form of oxalate oxidase has been found to provide a less strong response to the presence of oxalic acid than the purified oxalate oxidase. The partially purified oxalate oxidase is available as product O4127 from Sigma-Aldrich of St Louis, USA.

Instead of using simple adsorption to attach the oxalate oxidase to the working electrode, coupling chemistry may be used. The coupling chemistry may for example use glutaraldehyde. Experiments have shown that the glutaraldehyde allows the oxalate oxidase to remain active. However, adsorption may provide better retention of oxalate oxidase on the electrode than glutaraldehyde.

In general, a number of different methods may be used to attach an enzyme (e.g. oxlate oxidase) to an electrode or to keep the enzyme adjacent to the electrode. For example, surface adsorption, with or without stabilisers, may be used. Physical entrapment, wherein the enzyme is kept in the vicinity of the electrode surface by attaching a permeable membrane over the top of the electrode, may be used. The membrane may be cellulose acetate, collagen, polycarbonate or general purpose dialysis tubing. Polymer entrapment, wherein a polymer is deposited electrochemically on the surface, may be used, the enzyme being entrapped in the polymer or subsequently covalently or electrostatically attached to the polymer. Covalent binding, for example gold-thiol bonds formed between enzyme cystein residues and a gold electrode, may be used. Immobilisation via lysine residues, for example using carbodiimide or N-hydroxysuccinimide mediated coupling, may be used.

The working electrode 6 may be formed from carbon paste and the reference electrode 16 may be formed from a 60:40 combination of silver and silver chloride paste. The reference electrode 16 provides a stable reference equilibrium potential which may be used as a stable reference point against which the potential at the working electrode 6 may be measured. The reference electrode may partially encircle the working electrode. The pathogen sensor 1 may have an electrode configuration which includes a counter electrode (e.g. formed from carbon paste) in addition to the reference electrode. The sensor may for example comprise sensor BE2050824D1 which is available from Gwent Electronic Materials Ltd of Pontypool, United Kingdom.

The carbon paste of the working electrode 6 includes Prussian blue (ferric hexacyanoferrate) which acts as a mediator (the oxidised form of Prussian blue being used to pre-oxidise the working electrode 6). The oxidised form of Prussian blue catalyses the reduction of hydrogen peroxide at the working electrode 6 (it acts as an artificial peroxidise) and allows detection of hydrogen peroxide at significantly lower potentials than would be the case in the absence of a mediator (e.g. it allows detection at less than 0.6 volts). Applying a lower potential to the working electrode in this manner is advantageous because it reduces the detection of other electroactive species, thereby increasing the accuracy with which hydrogen peroxide is detected.

The nutrient liquid 8 may for example contain potato dextrose broth. The nutrient liquid may for example be obtained by mixing 1% of potato dextrose broth with a minimal salt solution (i.e. a solution containing inorganic salts). Other concentrations of potato dextrose broth may be used. The minimal salt solution, which may also be referred to as minimal media, may for example be a recipe in the literature and made up as: 1000 mg/L (NH4)2SO4; 500 mg/L K2HPO4; 500 mg/L KH2PO4; 450 mg/L NaCl; 250 mg/L MgSO4.7H2O; 5 mg/L Na-NTA; 0.5 mg/L FeCl3.6H2O; 0.5 mg/L CuSO4.5H2O; 0.5 mg/L ZnCl2; 0.5 mg/L MnSO4.H2O; 0.5 mg/L Na2MoO4.2H2O and pH adjusted to pH 5 using 1M HCl). The minimal salt solution may alternatively be M9 minimal salts, available from BD of New Jersey, USA. Other minimal salt solution may be used.

It is known from the published literature that potato dextrose based nutrients promote the growth of S. sclerotiorum and the production of oxalic acid by S. sclerotiorum. Published papers which mention growth of S. sclerotiorum and the production of oxalic acid in potato dextrose based nutrients include:
“Mycelial growth and production of oxalic acid by virulent and hypovirulent isolates of Sclerotinia sclerotiorum”; T Zhou and G J Boland; Can. J. Plant. Pathol. 21: 93-99 (1999);
“Oxalic acid production and its role in pathogenesis of Sclerotinia sclerotiorum”; P Magro, P Marciano and P Di Lenna; FEMS Microbiology Letters 24 (1984) 9-12;
“Oxalic Acid, a Pathogenicity Factor for Sclerotinia sclerotiorum, Suppresses the Oxidative Burst of the Host Plant”; S G Cessna, V E Sears, M B Dickman and P S Low; The Plant Cell, Vol. 12, 2191-2199, November 2000;

Nutrient liquid containing potato dextrose broth has been found to be effective in promoting growth of S. sclerotiorum and promoting production of oxalic acid by S. sclerotiorum. For example, growth of S. sclerotiorum and production of oxalic acid by S. sclerotiorum has been seen in a nutrient liquid containing 2.4% potato dextrose broth.

When the pathogen sensor is in use, the nutrient liquid 8 provides nutrients which allow S. sclerotiorum to grow in the nutrient liquid. Nutrients used by the S. sclerotiorum over time may be replaced from a nutrient reservoir (not shown), for example in the manner described further above in connection with FIG. 1. After growing in the nutrient liquid 8 for a period of time, the S. sclerotiorum produces oxalic acid. The catalytic activity of the oxalate oxidase 20 with the oxalic acid generated by the S. sclerotiorum (and with oxygen) causes the generation of hydrogen peroxide at the working electrode 6 along with carbon dioxide. As described above, the presence of the hydrogen peroxide at the working electrode 6 is detected by applying a potential to the working electrode and then measuring a current generated by reduction of the hydrogen peroxide at the working electrode. The reduction of hydrogen peroxide at the working electrode is catalysed by the Prussian blue in the electrode.

The potential applied to the working electrode 6 is stepped between a first value at which no electroactive reduction of the hydrogen peroxide occurs and a second value at which electroactive reduction of the hydrogen peroxide occurs. The potential step may for example be applied intermittently. The potential may for example be stepped between 0 volts and around 0.6 volts (or lower). The value of the potential applied to the working electrode 6 may be measured relative to the reference electrode 16. The change of potential at the working electrode 6 causes a characteristic charging and decay current which is proportional (e.g. directly proportional) to the concentration of the hydrogen peroxide at the electrode surface. The resulting current is monitored by measurement electronics (not shown) which identify the presence of oxalic acid based on the monitored current, and which thereby identify the presence of S. sclerotiorum in the nutrient liquid 8.

An experiment has been performed using the sensor described above (without potato dextrose broth) to confirm that the sensor electrochemistry is capable of detecting the presence of oxalic acid. The working electrode 6 and the reference electrode 16 were covered with 100 μl of electrolyte (e.g. 50 mM succinic acid 100 mM KCl pH 3.8 buffer). Oxalic acid was then added to the electrolyte such that the concentration of the oxalic acid increased gradually. The electrochemical measurement was carried out by applying a potential of −0.1 V to the working electrode (measured relative to the reference electrode) for 50 seconds and measuring the resulting current. The current after 40 seconds was recorded and plotted in a graph as a function of oxalic acid concentration. The results are shown in FIG. 3, both for the purified form of oxalate oxidase and the partially purified form of oxalate oxidase. In FIG. 3 squares indicate data obtained using the purified form of oxalate oxidase, and diamonds indicate data obtained using the partially purified form of oxalate oxidase. As may be seen from FIG. 3, for both types of oxalate oxidase the size of the measured current increases significantly as the concentration of oxalic acid is increased. The slope of the graph is downwards because the current is a negative current (the magnitude of the current increases). As may be seen from FIG. 3, purified oxalate oxidase provided a stronger response than partially purified oxalate oxidase. These results confirm that the pathogen sensor described above may be used to detect oxalic acid.

Experiments have also been performed using the sensor described above, with various different liquid nutrient media being provided over the electrodes 6, 16 (the nutrient media are listed below). The liquid nutrient media were prepared as a 1% w/v solution in minimal media pH 5 (the minimal media is from a recipe in the literature and made up as: 1000 mg/L (NH4)2SO4; 500 mg/L K2HPO4; 500 mg/L KH2PO4; 450 mg/L NaCl; 250 mg/L MgSO4.7H2O; 5 mg/L Na-NTA; 0.5 mg/L FeCl3.6H2O; 0.5 mg/L CuSO4.5H2O; 0.5 mg/L ZnCl2; 0.5 mg/L MnSO4.H2O; 0.5 mg/L Na2MoO4.2H2O and pH adjusted to pH 5 using 1M HCl). 25 mM glucose was also added to promote Sclerotinia growth. The pH was further adjusted to 3.8 before the experiment was performed. This was done because it is expected that the pH of the nutrient medium will drop after fungal growth and oxalic acid production by S. sclerotiorum. Furthermore, 3.8 may be the optimum pH for activity of the oxalate oxidase. In addition, the electrochemistry used by the pathogen sensor is more effective at more acidic pH than at less acidic pH.

For each liquid nutrient, increasing amounts of oxalic acid were added to the liquid nutrient such that the concentration of the oxalic acid increased gradually. The electrochemical measurement was carried out by applying a potential of −0.1 V to the working electrode (measured relative to the reference electrode) for 50 seconds and measuring the resulting current. The current after 40 seconds was recorded and plotted in a graph as a function of oxalic acid concentration. Results from the experiment are shown in FIG. 4, which is a graph which shows the detected current as a function of oxalic acid concentration for a variety of different liquid media. The media are labelled in FIG. 4 as follows:
E45—50 mM succinic acid 100 mM KCl pH 3.8
E57—1% potato dextrose broth minimal media pH 3.8
E58—1% Yeast nitrogen base without amino acid minimal media pH 3.8
E59—1% YPD broth in minimal media pH 3.8
E60—1% sabouraud dextrose liquid medium in minimal media pH 3.8
E43—1% soytone in minimal media pH 3.8
E61—1% czapek dox liquid medium in minimal media pH 3.8
E62—1% yeast tryptone broth in minimal media pH 3.8
E63—1% LB Lennox broth in minimal media pH 3.8
E64—1% yeast extract in minimal media pH 3.8
E65—1% mycological peptone in minimal media pH 3.8
E66—1% tryptone soya broth in minimal media pH 3.8
E67—1% beef extract in minimal media pH 3.8
E68 1% granulated tryptone in minimal media pH 3.8

As may be seen from FIG. 4, some nutrient media provide a significantly increased current as the concentration of oxalic acid increases. These are: 1% potato dextrose broth minimal media pH 3.8, 1% sabouraud dextrose liquid medium in minimal media pH 3.8, 1% Yeast nitrogen base without amino acid minimal media pH 3.8, and 1% czapek dox liquid medium in minimal media pH 3.8. 50 mM succinic acid 100 mM KCl pH 3.8 and 1% YPD broth in minimal media pH 3.8 also provide an increased current as the concentration of oxalic acid increases, but the increase is significantly less.

As noted further above, it is known from the published literature that potato dextrose based nutrients promote the growth of S. sclerotiorum and the production of oxalic acid by S. sclerotiorum. Since potato dextrose broth provides growth of S. sclerotiorum and oxalic acid production, and provides a strong current increase as oxalic acid concentration increases, potato dextrose broth may be used in the pathogen sensor to detect S. sclerotiorum. Potato dextrose broth is preferred over potato dextrose agar because the detection of oxalic acid in a liquid medium is significantly easier than detection of oxalic acid in a solid medium such as a gel.

It has been found via experimentation that Czapek dox does not promote growth of S. sclerotiorum and oxalic acid production by S. sclerotiorum. Czapek dox should therefore not be used in the pathogen sensor when monitoring for S. sclerotiorum.

Sabouraud dextrose liquid medium is expected to promote growth of S. sclerotiorum and oxalic acid production by S. sclerotiorum.

Other media provide little or no increased current as the concentration of oxalic acid increases, because they interfere with the electrochemistry of oxalic acid detection. Carbohydrate based media (such as potato dextrose based media) may give rise to little or no interference with the electrochemistry of oxalic acid detection. However, soytone based media inhibit oxalate oxidase on the electrode, therefore interfering with the enzyme mediated electrochemical detection.

Alternative embodiments of the invention are shown in FIGS. 5-9. In FIG. 5 the working electrode 26 comprises carbon paste without a mediator. Some features of the embodiment shown in FIG. 5 correspond with those of the embodiment shown in FIG. 2 and are provided with the same reference numerals. This embodiment of the invention may require a higher voltage to be applied in order to detect the presence of hydrogen peroxide (compared with the case when a mediator such as Prussian blue is present in the electrode). A potential drawback of the embodiment shown in FIG. 5 is that in addition to hydrogen peroxide, reduction reactions may also generate other electroactive species in the liquid 8. These other electroactive species may modify the current measured from the working electrode 6 and this may give rise to erroneous results.

Some fouling of the electrode may occur. In this context fouling may refer to proteins and other chemical species being non-specifically adsorbed at the working electrode 26. Adsorbed proteins or other chemical species may form a layer on the working electrode 26 which inhibits diffusion of electrons or ions at the electrode, thereby limiting the reduction of the hydrogen peroxide (and thereby limiting the current generated as a result of the oxalic acid produced by the S. sclerotiorum). One way in which fouling may be minimised or avoided is by keeping the liquid away from the electrode until a measurement is to be performed (as described further below in relation to FIG. 10).

It may be possible to prevent interfering species from reaching the working electrode 6 using pre-oxidation (e.g. with metal oxides), thereby improving the accuracy with which the hydrogen peroxide concentration is measured. An oxidant may for example be provided as nanoparticles which are interspersed on the electrode surface with the oxalate oxidase 20, or may for example be provided as a layer which lies over the oxalate oxidase, or may for example be provided in a multilayer stack which alternates between the oxidant and the oxalate oxidase. The oxidant catalyses the oxidation of interfering electroactive species into chemically inert forms before they reach the electrode 6. This prevents or reduces the detection of interfering species at the electrode 6.

In an alternative embodiment, an ion selective membrane may be provided above the oxalate oxidase, the ion selective membrane active to prevent or restrict interfering species from reaching and reacting with the oxalate oxidase. FIG. 6 shows this schematically in cross-section. Some features of the embodiment shown in FIG. 6 correspond with those of the embodiment shown in FIG. 5 and are provided with the same reference numerals. A membrane or gel layer 11 is provided over the liquid growth media 9. The membrane or gel layer 11 (and optionally the liquid growth media 9) may be considered to be a growth medium upon which and/or within which a pathogen may grow. An ion selective membrane 22 is provided in the liquid growth media 9. The ion selective membrane 22 prevents or restricts interfering species from reaching and reacting with the oxalate oxidase 20 but allows oxalic acid to reach and react with the oxalate oxidase.

Although some illustrated embodiments of the invention do not include a membrane or gel layer over the liquid growth media, a membrane or gel layer may be provided in connection with any embodiment. The membrane or gel layer may for example provide a surface upon which and/or within which the S. sclerotiorum (or other pathogen) may grow. However, a membrane or gel layer is not needed; the S. sclerotiorum (or other pathogen) may grow in a liquid nutrient without a membrane or gel layer.

Although illustrated embodiments of the invention comprise a liquid growth media, a gel growth media may be used instead of the liquid. The gel may be kept hydrated using a reservoir of fluid. For example, the gel may be kept hydrated using a reservoir of water based nutrients as described further above in relation to FIG. 1. Keeping the gel hydrated avoids the possibility that the growth of S. sclerotiorum on the gel is inhibited by the gel being dry. In addition, it facilitates detection of oxalic acid produced by the S. sclerotiorum. If the gel is not hydrated then oxalic acid produced by the S. sclerotiorum may not diffuse freely to the oxalate oxidase. In addition, dehydration of the gel could destabilise or denature the oxalate oxidase. Dehydration could also prevent the flow of electrons and ions between the working electrode and the reference electrode, thereby restricting electrochemical detection of the hydrogen peroxide.

FIG. 7 shows a further alternative embodiment of the invention in cross-section. In this embodiment the oxalate oxidase 20 is immobilised in a biocompatible polymer 28. Other features of this embodiment correspond with those shown in FIG. 5 and are provided with the same reference numerals. The biocompatible nature of the polymer allows the oxalate oxidase 20 to be retained in the vicinity of the working electrode 26 in its active form. The biocompatible polymer 28 may for example be a conducting polymer such as polyaniline, mucin/chitosan (mucin—a high molecular weight, heavily glycosylated protein (glycoconjugate)/chitosan—a linear polysaccharide composed of randomly distributed β-(1-4)-linked D-glucosamine (deacetylated unit) and N-acetyl-D-glucosamine (acetylated unit)), mucin/Carbapol®, (Carbopol® is polymers commonly used as thickeners, suspending agents and stabilizers available from Lubrizol limited) or any other suitable polymer. The polymer may also be a hydrogel such as polymethylmethacrylate. The biocompatible polymer 28 and immobilised oxalate oxidase 20 may be provided as a polymer film (e.g. a thick polymer film) on the working electrode 26.

The biocompatible polymer 28 may help to confer stability to the oxalate oxidase 20. In addition, it may block the electrode 6 from fouling by unwanted electroactive species. This is because the biocompatible polymer 28 provides a steric barrier which prevents proteins and oxidising species from being able to approach the surface of the working electrode 6. Prevention of fouling using the biocompatible polymer may be particularly beneficial because the pathogen sensor 1 may be operated over a considerable period of time (e.g. 10 hours or more, 24 hours or more, 2 days or more, or 4 days or more), during which time an accumulation of proteins and oxidising species at the working electrode 6 could lead to a significant loss of sensitivity at the working electrode (and could also lead to interfering background signals).

As mentioned above, the biocompatible polymer 28 may be a hydrogel such as a methyacrylate based polymer. The methacrylate containing biocompatible polymer may be formed by providing a thick film of polyglycerol monomethacrylate (PGMMA) on the working electrode 6, then polymerising and reacting the PGMMA with the oxalate oxidase through NHS-EDC coupling chemistry (e.g. as described in Bioconjugate Techniques by G. T. Hermanson (1996)). This provides a thick biocompatible polymer. The thickness of the PGMMA may be controlled by selecting an appropriate thickness for the pre-polymerised film.

A further alternative embodiment is shown in FIG. 8. The embodiment shown in FIG. 8 corresponds with that shown in FIG. 7, except that the working electrode 6 comprises a mediated carbon electrode (mediation being provided for example by Prussian blue). The mediated carbon working electrode 6 inhibits or restricts the detection of electroactive species other than hydrogen peroxide, as explained above in relation to FIG. 2. Other features of this embodiment correspond with those shown in previously described figures and are provided with the same reference numerals.

A further alternative embodiment of the invention is shown in FIG. 9. The embodiment shown in FIG. 9 corresponds with that shown in FIG. 7, except that the biocompatible polymer 28 is provided with horseradish peroxidase 30 in addition to oxalate oxidase 20 (it is a bienzyme system). Other features of this embodiment correspond with those shown in FIG. 5 and are provided with the same reference numerals. The horseradish peroxidase 30 is a secondary enzyme which catalyses the reduction of hydrogen peroxide and therefore allows detection of the presence of S. sclerotiorum using a lower applied potential at the working electrode 26 (compared with the potential used for direct detection). This may provide improved selective detection of the hydrogen peroxide, since using a lower potential reduces the detection of other electroactive species. The embodiment shown in FIG. 9 may however be more expensive to produce than other embodiments due to its increased complexity.

The biocompatible polymer 28 may be used to immobilise an enzyme other than oxalate oxidase or horseradish peroxidase.

Although the embodiment shown in FIG. 9 provides the oxalate oxidase 20 and horseradish peroxidase 30 in a biocompatible polymer 28, the oxalate oxidase and horseradish peroxidase may be provided in other ways. For example the oxalate oxidase and horseradish peroxidase may be provided on the surface of the working electrode 6.

Components of different embodiments of the invention may be combined with one another. For example, a mediated working electrode may be used in any of the illustrated embodiments of the invention.

The above described embodiments provide immobilisation of an enzyme (e.g. oxalate oxidase) or enzymes (e.g. oxalate oxidase and horseradish peroxidase) in the vicinity of an electrode 6, 26. In this context the term ‘in the vicinity’ may be interpreted as meaning sufficiently close that electroactive species (e.g. hydrogen peroxide) generated due to the presence of oxalic acid and the enzyme may be efficiently detected using the electrode. If the nutrient were to be a gel, and the enzyme were to be located too far from the electrode 6 then the electroactive species generated due to the presence of the oxalic acid would have little or no reaction with the electrode (the reaction rate will be limited by diffusion kinetics in the gel 8). As a result the presence of the electroactive species might not be detected. In these circumstances, moving the enzyme closer to the electrode 6 will increase the strength of the reaction of the electroactive species with the electrode, and increase the strength of an output provided from the electrode. Thus, it may be advantageous to provide the enzyme on the electrode surface or adjacent to the electrode surface (the term ‘in the vicinity of the electrode’ is intended to encompass both of these possibilities). Since diffusion kinetics also apply in a liquid, it is also advantageous to provide the enzyme on the electrode surface or adjacent to the electrode surface in a nutrient liquid.

The immobilisation of the oxalate oxidase (and/or other enzymes) may be done in a manner which allows the oxalate oxidase to retain activity and stability, and which may prevent or inhibit the oxalate oxidase from leaching out from its initial position, and may prevent or inhibit the oxalate oxidase from denaturing. For example, the oxalate oxidase may be provided on the electrode in the manner described further above. In embodiments in which the oxalate oxidase is provided on the electrode, modification of the surface of the electrode by the oxalate oxidase should not adversely affect diffusion of hydrogen peroxide and electrons between the oxalate oxidase and the electrode.

When providing the oxalate oxidase (and/or other enzymes) on the electrode, the electrode may be treated in order to facilitate a more homogeneous deposition of the oxalate oxidase. Binder chemicals which may be used when printing the electrode may make the electrode surface quite hydrophobic. This may make it difficult to achieve regular homogeneous oxalate oxidase (and/or other enzyme) deposition on the electrode surface. This may lead to loss of activity or sensitivity. To overcome this the electrode surface may be modified by detergents such as Triton X-100 or Brijj-30, thereby facilitating an even distribution and adsorption of the oxalate oxidase (and/or enzymes). Other treatments may be applied to the electrode surface such as plasma treatment (plasma is a partially ionized gas which has enough energy to ionize other atoms e.g. the atoms on the electrode surface thus changing the surface chemistry), or electrochemical pre-treatment of the working electrode.

The working electrode 6, 26 shown in FIGS. 2 to 7 is formed from carbon paste (which may be mixed with a mediator such as Prussian blue). Electrodes formed from carbon paste may be produced at low cost (compared with electrodes formed using some other materials) and may be relatively easy to form using mass production techniques. The carbon electrodes may for example include Prussian blue or cobalt phthalocyanine, which may allow the electrode to selectively sense hydrogen peroxide (i.e. excluding other electroactive species).

In an alternative embodiment, the electrode may be formed from indium tin oxide (ITO), for example on a glass slide which acts as a substrate. A disadvantage of using an ITO electrode is that it may not be compatible with the detection of hydrogen peroxide unless it is pre-treated. This is because differences in the surface chemistry and properties of ITO (compared with for example carbon paste) may cause reduction of atmospheric oxygen to occur at the working electrode. This reduction of atmospheric oxygen may for example occur when the working electrode is held a potential which is used to detect the presence of hydrogen peroxide (e.g. −0.6 volts), and will add to a noise signal at the electrode.

A pre-treatment may be applied to an ITO electrode in order to allow it to detect hydrogen peroxide reduction without generating a large noise signal due to atmospheric oxygen reduction. The pre-treatment may comprise modifying the surface of the ITO electrode by applying high voltages to it (e.g. as described in X. Cai, B. Ogorevc, G. Tavcar and J. Wang, Indium-tin oxide film electrode as catalytic amperometric sensor for hydrogen peroxide. Analyst 120 (1995), pp. 2579-2583). A disadvantage of pre-treating the ITO electrode is that it may add considerable complexity to the manufacture of the pathogen sensor.

In an alternative approach, instead of pre-treating an ITO electrode, horseradish peroxide may be provided at the ITO electrode in combination with an oxalate oxidase. This may be done for example using the arrangement shown in FIG. 9 or may be done for example by providing the horseradish peroxidase and the oxalate oxidase on the electrode. The horseradish peroxidase acts as a secondary enzyme which catalyses the reduction of hydrogen peroxide at the electrode. This may allow electrochemical detection of hydrogen peroxide to be performed using an ITO electrode at a more neutral applied potential (e.g. less negative than −0.6 volts).

Additionally or alternatively, Prussian blue may by provided at the ITO electrode. As explained above, the Prussian blue acts as an artificial peroxidise which catalyses the reduction of hydrogen peroxide. Again, this may allow electrochemical detection of hydrogen peroxide to be performed using an ITO electrode at a more neutral applied potential (e.g. less negative than −0.6 volts).

In general, Prussian blue may be combined with a variety of different electrode materials, including carbon paste, glassy carbon, graphite, carbon nanotubes, platinum, silver, silver chloride, gold and ITO. When Prussian blue is used the detection limit for hydrogen peroxide may be in the micromolar range. Prussian blue may be deposited onto electrodes using a variety of techniques including electrochemical and chemical methods, and may also be deposited as nanoparticles. Carbon electrodes which include Prussian blue or cobalt phthalocyanine are commercially available and may for example be purchased from Gwent Electronic Materials of Pontypool, United Kingdom. Although Prussian blue is less stable at alkaline pH values compared with acidic pH values, this may not be a disadvantage for the pathogen sensor because the gel 8 may be optimised at acidic pH values.

Other biochemical and/or chemical elements which decrease the electrochemical sensing potential of the electrode needed for an electroactive species to be detected (e.g. hydrogen peroxide) may be used instead of Prussian blue as a mediator which mediates the electrode. For example cobalt phthalocyanine may be used. Cobalt phthalocyanine electrodes detect hydrogen peroxide at around +0.5 V; less that the potential required to detect hydrogen peroxide on bare carbon electrodes. The detection of hydrogen peroxide using cobalt phthalocyanine electrodes is described in: Crouch, E., Cowell, D. C., Hoskins, S., Pittson, R. and Hart, J. P. (2005). Amperometric, screen-printed, glucose biosensor for analysis of human plasma oxidase using a biocomposite water-based carbon ink incorporating glucose oxidase. Analytical Biochemistry, 14, 17-23

At higher applied potentials (e.g. around +0.7 V), cobalt phthalocyanine will react directly with oxalic acid to produce a current. This is described in Li and Guarr (1991) Electrocatalytic oxidation of oxalic acid at electrodes coated with polymeric metallophthalocyanines. Journal of Electroanalytical and Interfacial Electrochemisrry, 317, 189-202). Consequently, oxalic acid may be measured directly without the need for an enzyme. However, an advantage of using an enzyme is that when an enzyme is used the electrochemical reaction occurs at a lower overpotential, thereby reducing the risk of unwanted currents being generated from other electroactive species present in the assay. A more sensitive measurement was obtained using oxalate oxidase on a Prussian blue mediated carbon electrode than was obtained using direct detection via a cobalt phthalocyanine electrode.

Any suitable mediator may be used to mediate an electrode of the pathogen sensor. Mediators which could be used instead of Prussian blue (potassium hexacyanoferrate) or cobalt phthalocyanine include Quinones, Ferrocene, Ferrocyanide, Methylene green, Osmium complexes e.g. osmium polypyridyl, Polypyrrol, Ruthenium complexes, and Pthalocyanines (i.e. pthalocyanines other than cobalt phthalocyanine).

The mediator may be freely diffusible to shuttle electrons between the enzyme and electrode surface. The mediator may be tethered to the enzyme and electrode. Tethered mediators are sometimes described as ‘wired’ enzymes. A conducting polymer such as polypyrrole and glucose oxidase is an example of a wired enzyme system.

The mediator may be used with redox enzymes (such as horseradish peroxidase) which depend on the activity of co-substrates which require high overpotentials for regeneration of the redox active co-substrate species.

The electrode may for example be modified by a biochemical and/or chemical recognition element. This may for example include incorporating an enzyme, antibody, DNA or chemical species into the electrode which may enhance or change the electrochemical response of the electrode.

The electrode may be formed from carbon, including screen printed carbon, glassy carbon, carbon nanotubes, graphene, carbon fibre, pyrolytic graphite carbon, metallised carbons e.g. platinised carbon. The electrode may be formed from composite materials composed of a powdered electronic conductor e.g. carbon powder or carbon nanotubes, and a binding agent such as polymeric material or paste. The electrode may be formed from indium tin oxide, platinum, silver, silver chloride, nickel, iron, copper, mercury (including mercury amalgams), palladium, iridium, or gold. Forming the electrode from gold may be relatively costly and in addition may not be compatible with a biocompatible polymer in which the oxalate oxidase may be provided. In general, the electrode may be formed from any suitable material which conducts electrons.

As explained above, horseradish peroxidase catalyses the reduction of hydrogen peroxide and allows hydrogen peroxide produced from the oxalic acid to be detected at lower electrochemical potentials (compared with direct electrochemical sensing of hydrogen peroxide). Since horseradish peroxidase is a redox enzyme, it may be beneficial to connect it to the surface of the working electrode 6 either directly (to allow direct electron transfer) or indirectly using mediators such as ferrocene (to allow the catalytic cycle to proceed and reduce hydrogen peroxide). In general, direct electron transfer methods using horseradish peroxidase may not be ideal for biosensing applications, because the horseradish peroxidase may denature at the electrode surface with the consequence that electron transfer rates between the electrode and the active sites of the horseradish peroxidase become slow. Mediators may be used to overcome slow heterogeneous electron transfer rates between the electrode and horseradish peroxidase. The mediator should be freely diffusible between the horseradish peroxidase and the electrode surface. It may be desirable that the mediator has high heterogeneous electron transfer rates and high reactivity with horseradish peroxidase. The mediator may be selected to not cross-react or inhibit the oxalate oxidase. In general, materials included in the pathogen sensor may be selected to not co-react with horseradish peroxidase.

The horseradish peroxidase may be applied such that it overlaps beyond edges of the oxalate oxidise. This reduces the likelihood that the horseradish peroxidase has a spatially limited activity which does not truly reflect the activity of the oxalate oxidase.

Oxalate oxidase and horseradish peroxidase have different optimal pH values. The optimal pH for oxalate oxidase is 4 and the optimal pH for horseradish peroxidase is 7. If oxalate oxidase and horseradish peroxidase are used, the pH in the pathogen sensor may for example be selected to be a value which lies between these two values, that is, between pH 4 and pH 7. More preferably the pH range is selected to be between pH 4.5 and 6.5. The pH in the pathogen sensor may be neutral, as this may encourage S. sclerotiorum growth. The pH of the pathogen sensor may change during the lifetime of the pathogen sensor, for example becoming more acidic due to accumulation of oxalic acid produced by the S. sclerotiorum. The pH dependence of the enzyme activity (e.g. oxalate oxidase and horseradish peroxidase) may be modified by using enzymes from different sources, or by using genetic engineering techniques to produce enzymes which have a wider pH tolerance or optimal activity at a desired pH value.

Although above described embodiments of the invention monitor for the presence of hydrogen peroxide at an electrode, alternative embodiments of the invention may monitor for the presence of other electroactive species at an electrode.

The above described embodiments of the pathogen sensor use electrochemical transduction (i.e. the conversion of chemical energy into electrical energy) to detect the presence of oxalic acid. An advantage of using electrochemical transduction to detect oxalic acid is that it allows the pathogen sensor to be made relatively small, and allows it to be made using mass manufacturing techniques at relatively low cost (compared to making a pathogen sensor which uses other transduction methods).

In the embodiments shown in FIGS. 2 to 7 wires 18 extend downwardly from the working electrode 6 and the reference electrode 16, and pass through openings in the substrate 14 to measurement electronics. Similarly, in the embodiment shown in FIG. 1 a cylindrical channel 10 extends downwardly from the working electrode 6 to accommodate wires which pass to measurement electronics. Wires may however travel along other routes to measurement electronics. For example, wires may pass along the top of a substrate of the pathogen sensor. Where this is done a layer of insulation (e.g. a plastic layer) may be provided over the wires to insulate them from the electrode.

A sensor apparatus 140 which includes a plurality of pathogen sensors 100a-f according to an embodiment of the invention is shown schematically in FIG. 10. The pathogen sensors 100a-f are provided on a flexible tape 101. The flexible tape 101 may be provided with, for example, around one hundred pathogen sensors, and may be wrapped around a reel (not shown). A lead end of the flexible tape 101 may be connected to a second reel (not shown), which may be driven such that over time the flexible tape is unrolled from the reel and is rolled onto the second reel. The second reel may be driven such that every 24 hours the pathogen sensors are moved by a distance which corresponds to the separation between pathogen sensors (this movement is indicated by the arrow 102 in FIG. 10). The position of the second reel, and hence the positions of the pathogen sensors 100a-f, may be controlled by a control apparatus (not shown). The control apparatus may also control operation of puncturing arms and measurement electronics (described below).

Each pathogen sensor 100a-f may comprise a housing which is generally cylindrical (or has some other shape), and which is open at an upper end. The housing may for example have a depth of around 15 mm, and may for example have a diameter of around 6 mm. An impermeable barrier 104 may be located above the bottom of the housing, for example around 2 mm above the bottom of the housing, thereby defining a volume which is referred to hereafter as sampling volume 105. An electrode 106 is located at the bottom of the sampling volume 105. The electrode 106 may for example be provided with an oxalate oxidase, for example as described further above. Nutrient liquid 108 may be provided in the housing above the impermeable barrier 104. A film 106 (or other barrier) may be located above the nutrient liquid 108.

The pathogen sensor 100a may initially be located in a pre-sampling housing 109. A puncturing arm 110 in the pre-sampling housing 109 may be used to puncture the film 106. Following this, the pathogen sensor may be moved to a sampling location which is located outside of the pre-sampling housing. The sampling location is a location which receives air and airborne pathogens. In FIG. 10 pathogen sensor 100b is located at the sampling location.

The pathogen sensor 100b may remain at the sampling location for 24 hours, during which time pathogen spores may pass into the pathogen sensor. The pathogen spores may for example land in the nutrient liquid.

The pathogen sensor is then moved into an incubator 111. The incubator 111 has a temperature of 25° C., the temperature being selected to promote growth of the pathogen. Pathogen sensor 100c is shown in the incubator 109.

The pathogen sensor may be moved through the incubator 111 such that it is incubated for three days, as shown by pathogen sensors 100c-e in FIG. 10. The incubator may include some form of covering (not shown) for the pathogen sensors 100c-e which acts to prevent or inhibit evaporation of the nutrient liquid 108 from the pathogen sensors. Alternatively or additionally, the apparatus may include a liquid nutrient replenishment apparatus which is configured to periodically add liquid nutrient to the pathogen sensors 100c-e to replace evaporated liquid nutrient. During incubation the pathogen will grow and will release oxalic acid. The oxalic acid will mix with the nutrient liquid 108. During incubation the pathogen and the nutrient liquid are isolated from the electrode 106 by the impermeable barrier 104.

After the pathogen sensor has been incubated for three days, a puncturing arm 112 is used to puncture the impermeable barrier 104. This allows the nutrient liquid and oxalic acid to pass into the sampling volume 105 at the bottom of the pathogen sensor (as shown for pathogen sensor 100f). The nutrient liquid and oxalic acid thus come into contact with the electrode 106. Measurement electronics 113 are configured to apply a potential at the electrode 106, for example in the manner described further above. As described further above, the oxalic acid reacts with the oxalate oxidase to generate hydrogen peroxide which is detected by the electrode 106. This indicates that the pathogen has grown in the pathogen sensor.

The housing 103 of the pathogen sensor may be formed from a polymer. The polymer may include a coating which prevents or inhibits the release of volatile organics that could inhibit growth of the pathogen. The sampling volume 105 of the pathogen sensor may include a hydrophilic element which is arranged to draw the liquid nutrient and oxalic acid into the sampling volume. The sampling volume 105 may for example have a volume of 200 μl

An apparatus (not shown) which is arranged to draw air into the pathogen sensor may be provided at the top of the pathogen sensor.

Although the above description refers to sampling for 24 hours and incubating for 3 days before measurement, any suitable sampling and incubating periods may be used.

Incubation may for example be for between 3 and 7 days. The incubation may be at any suitable temperature. The temperature may for example be chosen to provide optimal growth of the pathogen. Any suitable number of pathogen sensors may be provided on the flexible tape 101. For example, sufficient pathogen sensors may be provided to allow pathogen sensing to be performed over an entire growing season.

Any suitable apparatus may be used to isolate a nutrient medium and a pathogen from an electrode during incubation of the pathogen. Similarly, any suitable apparatus may be used to end that isolation such that the nutrient medium and pathogen come into contact with the electrode when a measurement is to be performed. Keeping the nutrient medium and pathogen away from the electrode during incubation is advantageous because it avoids deterioration of the electrode that could occur if the nutrient liquid and pathogen were in contact with the electrode during incubation.

In an alternative arrangement, the pathogen sensors on the tape may not be pre-filled with liquid nutrient. The liquid nutrient may be delivered into the pathogen sensor after sampling takes place. The liquid nutrient may for example be delivered via a pump. The pump may be sterile, and the apparatus may include a washing apparatus arranged to wash the pump and keep it sterile.

The puncturing arms 110, 112 are examples of puncturing apparatus. The sensor apparatus 140 may include any suitable puncturing apparatus.

Each pathogen sensor 100a-f may be provided with an air sampling apparatus which is arranged to sample air and to direct spores from the air into the pathogen sensor. Such air sampling apparatus are well known in the art and are therefore not described here.

A sensor apparatus 40 which includes a pathogen sensor 1 according to an embodiment of the invention is shown schematically in FIG. 11. Features of the apparatus shown in FIG. 11 may be combined with features of the apparatus shown in FIG. 10. The sensor apparatus comprises a chamber 42 which has an opening (not shown) connected to the atmosphere at one end and has an opening (not shown) connected to a pump 44 at the other end. The opening connected to the pump may be larger than the opening connected to the atmosphere, such that a vacuum is generated in the chamber when the pump is operating. The sensor apparatus may be provided with a weather vane (not shown) and may be rotatably mounted such that it turns towards the wind. The sensor apparatus may include features described in Hirst J M (1951), An Automated Volumetric Spore Trap, Annals of Applied Biology, 39(2), pp 257-265, which is herein incorporated by reference.

The sensor apparatus includes a power supply unit 46 which comprises a power harvesting system 48 (for example a solar panel or wind turbine) which charges a battery 50. The battery 50 may be used to power electrical components of the sensor apparatus via a DC/DC converter 52. Other forms of power supply unit may be used.

In addition to the pathogen sensor 1, the sensor apparatus 40 may be provided with one or more additional ancillary sensors, for example in a meteorological unit 54. These may for example include one or more of a temperature sensor 56, a humidity sensor 58, a wind direction and wind speed sensor 60, a pressure sensor 62 and an ambient light sensor 64.

The sensor apparatus may be provided with control electronics 66, which may for example comprise a CPU. The measurement electronics which are used to apply a potential step to an electrode of the pathogen sensor 1 and to detect a resulting current may form part of the control electronics 66 or may optionally be provided as a separate entity 68. In addition to receiving data from the pathogen sensor, the control electronics 66 may receive data from the additional ancillary sensors 54-64 (e.g. via a signal conditioner 65). The control electronics 66 may include a memory which stores data as a function of time. The control electronics may thus allow the quantity of the pathogen at the sensor to be tracked over a period of time. Analysis electronics may be provided as part of the control electronics, the analysis electronics being used to analyse data received from the pathogen sensor (and optionally from other sensors).

The duty cycle of the pump 44 and other components of the sensor apparatus may be actively managed by the control electronics 66, for example to take into account a power budget arising from a battery 50 of the sensor apparatus.

Although only one pathogen sensor 1 is shown in FIG. 8, a plurality of pathogen sensors 1 may be provided in a single sensor apparatus 40. For example, more than one pathogen sensor which is configured to detect a particular pathogen may be provided in the sensor apparatus. Where this is done, a first pathogen sensor may be used to monitor for the presence of the pathogen over a period of time until a supply of nutrients is exhausted or close to being exhausted (and/or the pathogen sensor is dehydrated), whereupon operation of a second pathogen sensor which is configured to detect the pathogen may be initiated. This may for example be achieved by removing a film from the second pathogen sensor. This may be an automated process performed by a sensor selector unit 69 which is controlled for example by the control electronics 66, or may be performed manually. Alternatively, the sensor apparatus 40 may be configured to expose a first pathogen sensor to the atmosphere for a predetermined period of time (e.g. until a nutrient supply is substantially exhausted and/or the pathogen sensor is dehydrated), then move a second pathogen sensor from a sealed container such that it is exposed to the atmosphere. This may be an automated process or may be performed manually. The first and second pathogen sensors (and possibly additional pathogen sensors) may be provided in a cartridge (not shown) which is removable from the sensor apparatus 40. This may be an automated process performed by a sensor selector unit 69 which is controlled for example by the control electronics 66, or may be performed manually. The cartridge may for example comprise a disk which may be rotated to expose a selected pathogen sensor to the atmosphere.

The measurement electronics 68 may monitor electrodes of a pathogen sensor 1 which is newly exposed to the atmosphere, and may cease monitoring electrodes of a pathogen sensor which has been replaced by the newly exposed pathogen sensor. This switch may be controlled by the control electronics 66.

Additionally or alternatively, auxiliary pathogen sensors which are configured to detect the presence of different pathogens may be provided in the sensor apparatus 40. The auxiliary pathogen sensors may for example be capable of detecting proteins secreted by interfering pathogens.

A wireless network may be provided which enables communication between the sensor apparatus 40 (e.g. via a wireless transceiver 70) and remotely located system analysis and control electronics (not shown). Alternatively, a wire-based network may be provided to enable this communication. The remotely located system analysis and control electronics may for example be a CPU. The system analysis and control electronics may receive data from a plurality of sensors apparatus. The system analysis and control electronics may control a plurality of sensor apparatus 40 by sending control signals to the sensor apparatus via the wireless network. The control signals may for example instruct that a pathogen sensor 1 which has reached the end of its life is replaced by a new pathogen sensor. Wireless communication between the sensor apparatus 40 and the system analysis and control electronics may for example use local area wireless network (Wi-Fi) transmitters and receivers and/or GSM transmitters and receivers. Communication may include one or more relay nodes.

The system analysis and control electronics may analyse pathogen sensor data from sensor apparatus spread over an area such as a field, a plurality of fields, a farm or some other area. The data analysis may incorporate data from the additional ancillary sensors of the sensor apparatus. The data analysis may identify progress of a pathogen across the area, and may provide a forecast of the progress of the pathogen. The data received by the system analysis and control electronics may include a degree of data-redundancy, and this may be used to identify outlier pathogen sensor measurements which may indicate failure or incorrect operation of a pathogen sensor. The data-redundancy may also facilitate improved interpolation of pathogen ingress between pathogen sensors.

Data from a plurality of system analysis and control electronics may be collected at a central data analysis system (for example collecting data from across a region, country or internationally). The data may be merged with data from more traditional agronomy data sources, such as meteorological data or crop data obtained by satellite imaging. The central data analysis system may use the merged data to deliver ground-truthed real-time maps of pathogen progress.

In the above described illustrated embodiments of the pathogen sensor, the nutrient liquid 8, 108 (or gel) acts as a growth medium upon which and/or within which the pathogen may germinate and grow, and provides nutrients which facilitate growth of the pathogen (the nutrients thus sustaining the pathogen in a similar way to nutrients that the pathogen would extract from a plant). Various properties of the pathogen sensor may be selected to mimic a plant or mimic particular conditions, such that a pathogen may germinate and grow and mediate an event which is to be detected. The pathogen may be S. sclerotiorum or may be some other pathogen. Properties of the pathogen sensor may be selected to mimic part of a plant (e.g. a leaf or a stem) upon which and/or within which the pathogen may grow.

As explained above, the pathogen sensor may for example be configured to detect S. sclerotiorum. Where this is the case the pathogen sensor provides a growth medium (e.g. the nutrient liquid 8) upon which and/or within which S. sclerotiorum may grow, and provides nutrients which nourish the S. sclerotiorum over a period of time which is sufficient to allow the S. sclerotiorum to generate oxalic acid. In addition, the nutrients facilitate the production of oxalic acid by the S. sclerotiorum. This facilitation of the production of oxalic acid may be achieved for example by providing nutrients which facilitate growth of S. sclerotiorum via metabolic pathways which provide more oxalic acid production than alternative metabolic pathways (the alternative metabolic pathways producing less oxalic acid). Selective fungicides, antibiotics or antimicrobials may be incorporated in the pathogen sensor to inhibit the growth of other microorganisms which may inhibit S. sclerotiorum growth and/or produce oxalic acid or some other interferent electroactive species.

The pathogen sensor 1 may be configured to detect a pathogen other than S. sclerotiorum. This may be achieved for example by providing nutrients in the growth medium which nourish the pathogen to be detected and allow it to grow. For example, Sclerotinia other than S. sclerotiorum may grow in a potato dextrose based medium. For example, Sclerotinia homeocarpa may grow in a potato dextrose agar or a potato dextrose broth, and may release oxalic acid as it grows—see “Oxalic Acid Production by Sclerotinia homoeocarpa: the Causal Agent of Dollar Spot” by R A Beaulieu; Senior Honors Thesis; The Ohio State University; June 2008. For example, Sclerotinia minor may grow and release oxalic acid in a variety of media, as described in “Oxalic Acid Production and Mycelial Biomass Yield of Sclerotinia minor for the Formulation Enhancement of a Granular Turf Bioherbicide” by S C Briere, A K Watson and S G Hallett; Biocontrol Science and Technology (2000) 10, 281-289, the disclosure of which is herein incorporated by reference. The media mentioned in that paper include potato dextrose broth (PDB, Difco Laboratories, Detroit, Mich.) at pH 6.0; PDB at pH 6.0 plus 56-mm sodium succinate (PDB-SS).

The nutrients may also facilitate the production of a detectable substance by the pathogen. A supply of nutrients may be provided from a nutrient reservoir (e.g. via a one-way membrane). The substance which is detected by the pathogen sensor 1 may be a chemical or biological agent (including for example organic acids, nucleic acids, proteins (e.g. enzymes), toxins, hormones, metabolites, peptides, carbohydrates or lipids).

The pathogen sensor may be considered to provide a two-step method of pathogen detection. The first step is growth of the pathogen on and/or in the growth medium, and the second step is production of a detectable substance by the pathogen after some growth of the pathogen has occurred.

Because it detects a substance produced by a pathogen (e.g. generation of oxalic acid in the case of S. sclerotiorum), the pathogen sensor provides a real-time indication of the presence of the pathogen as well as the viability of the pathogen. That is, the pathogen sensor differentiates between an active pathogen and a dormant or dead pathogen. Furthermore, in addition to detecting the presence of the pathogen, embodiments of the invention may also provide an indication of the quantity of pathogen at the pathogen sensor.

An aspect of the pathogen sensor which may facilitate growth of the pathogen on and/or in the growth medium is hydration of the growth medium. The growth medium may be kept hydrated for example by delivering fluid to the growth medium from a fluid reservoir (e.g. via a one-way membrane). The fluid reservoir may be separate from the growth medium (e.g. located away from the growth medium as shown in FIG. 1).

An aspect of the pathogen sensor which may facilitate growth of the pathogen on and/or in the growth medium is delivery of nutrients to the growth medium. Nutrients may be delivered to the growth medium from a nutrient reservoir (e.g. via a one-way membrane). The nutrient reservoir may be separate from the growth medium (e.g. located away from the growth medium as shown in FIG. 1).

The nutrient reservoir and the fluid reservoir may be the same reservoir. The nutrient may be provided in a fluid which keeps the pathogen hydrated.

The pathogen sensor may allow a pathogen to grow in a manner which is similar to the manner in which the pathogen would grow on a plant. The pathogen sensor may for example provide a favourable growth environment for the pathogen such that the pathogen will grow on/in the growth medium at a speed which is faster than the speed of growth of the pathogen on the plant (e.g. through incubation of the pathogen sensor). This allows the plant to be protected through the application of a fungicide or other measures which will prevent or restrict the growth of the pathogen. A crop which comprises the plant may for example be protected in this manner.

The pathogen sensor 1 may be configured to detect a fungal pathogen, for example a fungal pathogen which generates oxalic acid. This may be achieved for example by providing nutrients in the growth medium which nourish the fungal pathogen to be detected.

The nutrients may also facilitate generation of a detectable substance by the fungal pathogen. Selective fungicides, antibiotics or antimicrobials may be incorporated in the pathogen sensor to inhibit the growth of other fungicides or other microorganisms as appropriate which may inhibit growth of the fungal pathogen or other microorganisms and/or interfere with detection of a substance produced by the fungal pathogen (e.g. oxalic acid) or other microorganisms.

As mentioned above, the substance which is detected by the pathogen sensor may be a chemical or biological agent (including for example organic acids, nucleic acids, proteins (e.g. enzymes), toxins, hormones, metabolites, peptides, carbohydrates or lipids). In this context the term ‘organic acid’ may be interpreted as meaning a molecule that contains a carboxylic acid functional group. Embodiments of the invention detect the organic acid using electrochemical transduction (as described above). Other chemical or biological agents may also be detected using electrochemical transduction.

Embodiments of the invention include an enzyme with which a chemical or biological agent released by a pathogen interacts, the interaction leading to an electronically detectable signal. The interaction of the enzyme with the chemical or biological agent may comprise the enzyme binding to and subsequently reacting with the chemical or biological agent. Any suitable enzyme may be used. The interaction may lead to the generation of an electroactive molecule which may then be detected using an electrode. The interaction may lead to the generation of a molecule which is the substrate for subsequent interaction with an enzyme (e.g. a different enzyme) or other reactive molecule. This subsequent interaction may lead to the generation of an electroactive molecule which may then be detected using an electrode. In this context, although the interaction of the chemical or biological agent with the first enzyme does not directly generate an electroactive molecule it leads towards generation of an electroactive molecule. The interaction may be considered to lead indirectly to the generation of an electroactive molecule, and thus may be considered to lead indirectly to an electronically detectable signal. One or more additional enzyme interactions may take place before the electroactive molecule is generated. These additional enzyme interactions may also be considered to lead indirectly to the generation of an electroactive molecule.

The interaction of the chemical or biological agent released by a pathogen with the enzyme may cause a conformational change in the enzyme which is recognised by other elements in the pathogen sensor (e.g. other enzymes), and this may lead to the generation of an electroactive molecule (either directly or indirectly). The conformational change may cause the enzyme to accept a substrate already present in the growth medium (the substrate being something other than the chemical or biological agent). Interaction of this substrate to the enzyme may lead to the generation of an electroactive molecule (either directly or indirectly).

The electronic detection apparatus may detect the chemical or biological agent released by a pathogen using some other form of transduction. The electronic detection apparatus may detect the chemical or biological agent via enzymatic, immunoassay (antigen-antibody binding), spectroscopic or other biosensing techniques. The electronic detection apparatus may use the passage of the chemical or biological agent through a membrane (e.g. as described above in relation to FIG. 3). Acid release from a pathogen may for example be detected using an electronic detection apparatus which uses detection of swelling of a gel, electrochemical sensing or detection of a refractive index change or colour change. The electronic detection apparatus may for example detect protein secretions arising from pathogen growth using antibody/antigen binding resulting in an optical refractive index change, mass change on a surface acoustic wave device or resonant quartz crystal microbalance, or electrochemical sensing.

As mentioned above, properties of the pathogen sensor may be selected to mimic a plant or mimic particular conditions. Properties of the pathogen sensor may be selected to mimic part of a plant (e.g. a leaf or a stem) upon which and/or within which the pathogen may grow. One or more of lighting, humidity and/or moisture, pH conditions, orientation and temperature may be selected to mimic a plant or part of a plant, or to mimic particular conditions.

The pathogen sensor may be configured to take into account photo-inhibition or photo-promotion of a pathogen. The natural lighting conditions which support pathogen germination and growth may be mimicked at the growth medium of the pathogen sensor. This may for example be through exposing the sensor surface to ambient light which has passed through appropriate optical filters, through illuminating the sensor surface using a photo-emitter such as a semiconductor or polymer, or through exposing the growth medium to ambient lighting.

The pathogen sensor may be configured to take into account humidity and/or moisture conditions. Appropriate humidity conditions and/or dew build up for an extended period (e.g. 6-12 hours) may be necessary for an event mediated by the pathogen to take place. The growth medium of the pathogen sensor may comprise a hydrophilic gel and/or polymer which provides moisture for the pathogen. Additionally or alternatively, the pathogen sensor may include a one-way membrane configured to wick water from a reservoir to the growth medium (e.g. in a manner analogous to that described above in relation to FIG. 1).

The pathogen sensor may be configured to take into account pH conditions which support pathogen germination and growth. The pH of the growth medium of the pathogen sensor may be selected via the inclusion of hydrophilic gels and buffers in the pathogen growth medium. Additionally or alternatively, the pH of the growth medium may be controlled by providing the pathogen sensor with a one-way membrane configured to wick a buffer from a reservoir to the growth medium (e.g. in a manner analogous to that described above in relation to FIG. 1).

The growth medium of the pathogen sensor may be oriented to take into account the effect of gravity in supporting pathogen growth. For example, the growth medium may have an orientation which corresponds with a likely orientation of a part of a plant on which the pathogen will grow.

The growth medium of the pathogen sensor may be held at a temperature (or have a temperature variation applied to it over time) which supports germination and growth of the pathogen. The temperature of the growth medium may for example be controlled using a Peltier-effect heat pump or any other suitable temperature control apparatus.

Selective fungicides, antibiotics or antimicrobials may be incorporated in the pathogen sensor to inhibit the growth of other microorganisms which may inhibit growth of the pathogen to be detected and/or interfere with detection of an event mediated by the pathogen.

The sensor apparatus may incorporate air filtering, for example using a filter which is sized to exclude larger interfering pathogens or other sources of interferents.

Although the description of embodiments of the invention has focussed on detection of fungal pathogens, the invention may be used to detect other pathogens. Similarly, although the description of embodiments of the invention has focussed on pathogens which grow on plants, the invention may be used to detect pathogens which grow elsewhere (e.g. in the human body, in an animal body, in foodstuffs, in water, etc). In an embodiment, the pathogen sensor may be used to detect a pathogenic bacteria. In an embodiment the pathogen sensor may be used to detect a pathogen from the Burkholderia genus, for example Burkholderia glumae (e.g. in grain rot and seedling rot in rice), or Burkholderia pseudomallei (e.g. which causes the disease melioidosis). Burkholderia releases oxalic acid and may therefore be detected using the above described embodiments of the pathogen sensor. In general, the pathogen sensor may be used to detect pathogens in a variety of application areas, including for example: healthcare (e.g. Aspergillus niger, B. pseudomallei, Saccharomyces cerevisiae), animal health (e.g. Aspergillus niger), environmental monitoring (e.g. S. sclerotiorum, Fomitopsis palustris), food spoilage (e.g. Botrytus cinera), post harvest grain storage (e.g. Burkholderia glumae, Botrytus cinera), pre-harvest seedling storage (e.g. Burkholderia glumae, Botrytus cinera), materials protection (e.g. Fornitopsis palustris) and bio-security (e.g. Burkholderia pseudomallei)”. Properties of the pathogen sensor may be selected to mimic an entity upon which and/or within which the pathogen may grow.

Although embodiments of the invention have referred to the pathogen sensor being provided in a crop which is growing or adjacent to a crop which is growing, the pathogen sensor may be provided in other locations. For example, the pathogen sensor may be provided in a storage area in which a crop is stored after the crop has been harvested (e.g. a warehouse or barn).

References in this description to growth of the pathogen may be considered to include germination of the pathogen (the pathogen is metabolically active during germination and may thus be considered to be growing).

Detection of an electroactive species, as described in the above embodiments, is an example of detection via an electrochemical change. Other electrochemical changes which may be detected by embodiments of the invention may for example be a change of capacitance, inductance or some other electrical property. Embodiments of the invention may for example use antibody binding in conjunction with impedance spectroscopy detection to monitor for an event mediated by the pathogen (the electrochemical change in this case being a change of impedance).

Embodiments of the invention may be considered to use an enzyme system to mediate and monitor an electrochemical change from a chemical agent which is electroactive at a high applied potential (e.g. oxalic acid) to a chemical agent which is electroactive at a lower applied potential (e.g. hydrogen peroxide).

The term ‘growth medium’ as used in the above description may be interpreted as meaning any medium upon which and/or within which a pathogen may grow (the structure being sufficiently strong to support the pathogen). The growth medium may include any desired level of porosity. The growth medium may be a nutrient liquid. The term growth medium may be considered to mean an environment favourable to growth of a pathogen (the environment may be a liquid or a solid).

Features of any embodiment of the invention may be combined with features of any other embodiment of the invention.

Although some embodiments of the invention include a liquid nutrient medium, a solid nutrient medium such as a gel may be used instead of a liquid nutrient medium. An advantage of using a liquid nutrient medium is that diffusion of oxalic acid released by a pathogen will take place more readily in a liquid than in a solid, thereby allowing the oxalic acid to reach the electrode of the sensor more easily. A further advantage is that S. sclerotiorum grows more readily in a liquid nutrient medium than in a solid nutrient medium.

Features from different embodiments of the invention may be combined with one another.

1. A pathogen sensor comprising a growth medium upon which and/or within which a pathogen may grow, the growth medium comprising nutrients which facilitate growth of the pathogen, wherein the pathogen sensor further comprises an electronic detection apparatus configured to detect an electrochemical change mediated by the pathogen.

2. The pathogen sensor of claim 1, wherein the electrochemical change is caused by a chemical or biological agent produced by the pathogen.

3. The pathogen sensor of claim 2, wherein the chemical or biological agent is one of the following: an organic acid, a nucleic acid, a protein, an enzyme, a toxin, a hormone, a metabolite, a peptide, a carbohydrate or a lipid.

4. The pathogen sensor of claim 2, wherein the chemical agent is oxalic acid.

5. The pathogen sensor of claim 2, wherein the electronic detection apparatus comprises an enzyme that interacts with the chemical or biological agent, the interaction leading to an electronically detectable signal.

6. The pathogen sensor of claim 5, wherein the interaction generates an electroactive species or leads to the generation of an electroactive species, and wherein the electronic detection apparatus further comprises an electrode configured to detect the presence of the electroactive species.

7. The pathogen sensor of claim 6, wherein the enzyme is immobilised on a surface of the electrode.

8. The pathogen sensor of claim 6, wherein the enzyme is oxalate oxidase.

9. The pathogen sensor of claim 6, wherein the electrode is mediated with ferric hexacyanoferrate.

10. The pathogen sensor of claim 6, wherein the nutrients are separated from the electrode by a barrier which is configured to be punctured when detection of the electroactive species is to be performed.

11. The pathogen sensor of claim 1, wherein the growth medium is a liquid media which contains potato dextrose broth.

12. The pathogen sensor of claim 1, wherein the pathogen is a fungal pathogen.

13. The pathogen sensor of claim 1, wherein the pathogen is from the Sclerotinia species.

14. The pathogen sensor of claim 13, wherein the pathogen is Sclerotinia Sclerotiorum.

15. A sensor apparatus which comprises the pathogen sensor of claim 1 and further comprises measurement electronics configured to receive a signal from the electronic detection apparatus and to generate an output if the signal is indicative of an electrochemical change mediated by the pathogen.

16. The sensor apparatus of claim 15, wherein the sensor apparatus further comprises a control apparatus which is configured to expose the pathogen sensor to the air, incubate the pathogen sensor for a predetermined period of time, and then use the electronic detection apparatus to monitor for the electrochemical change.

17. The sensor apparatus of claim 15, wherein the sensor apparatus further comprises a puncturing apparatus configured to puncture a barrier which separates the growth medium from the electrode.

18. A method of detecting a pathogen comprising providing nutrients which facilitate growth of the pathogen on and/or in a growth medium for a period which is sufficiently long to allow a pathogen to mediate an electrochemical change, then using an electronic detection apparatus to detect the electrochemical change.

19. The method of claim 18, wherein the electrochemical change is caused by a chemical or biological agent produced by the pathogen.

20. The method of claim 19, wherein the chemical agent is oxalic acid.

21. The method of any of claim 18, wherein the electronic detection apparatus comprises an enzyme which interacts with the chemical or biological agent, the interaction leading to an electronically detectable signal.

22. The method of claim 21, wherein the enzyme is oxalate oxidase which catalyses the production of hydrogen peroxide from the oxalic acid.

23. The method of claim 18, wherein the growth medium is a liquid media which contains potato dextrose broth.

24. The method of claim 18, wherein the pathogen sensor is one of a plurality of pathogen sensors distributed over an area, and wherein the method comprises analysing outputs from the pathogen sensors to obtain information regarding the progress of the pathogen through the area.

25. The method of claim 18, wherein analysis of information provided from the pathogen sensor is combined with analysis of information provided from one or more sensors which sense one or more of: temperature, humidity, wind direction, wind speed, pressure sensor and ambient light.

26. (canceled)

27. (canceled)
